(19) United States
(12) Patent Application Publication
(10) Pub. No.: US 2004/0097521 A1
(43) Pub. Date: May 20, 2004

(54) Clinical treatment

(76) Inventors: Thomas Lundeberg, Lidingo (SE); Maria Concetta Romano, Rome (IT)

Correspondence Address: MERCHANT & GOULD PC, P.O. BOX 2903, MINNEAPOLIS, MN, 55402-0903, US

(21) Appl. No.: 10/450,724

(22) PCT Filed: Dec. 27, 2001
(86) PCT No.: PCT/SE01/02919

(30) Foreign Application Priority Data
Dec. 27, 2000 (SE) 0004867-8

(51) Int. Cl. A61K 31/496; A61K 31/198

(52) U.S. Cl. 514/255.01; 514/562

(57) ABSTRACT

The invention relates to the use of an effective dose of N-acetyl-L-cysteine, or a dimeric form thereof, for the manufacturing of a drug for reverting mammalian neoplasia cells of epithelial origin back to a normal differentiation whereby the abnormal proliferation of the neoplasia cells is reverted to a normal pathway.

The present invention refers the use of N-acetyl-L-cysteine, or a dimeric form thereof, for the clinical treatment of mammalian neoplasia cells of epithelial origin.

A tumor, or neoplasm, comprises new growth of tissue, in which the multiplication of cells is uncontrolled and progressive. Not all types of abnormal growth are malignant; those that are not are referred to as benign tumors. In contrast to malignant growths, benign tumors consist of an orderly growth of cells that often are identical to or very closely resemble their normal counterparts.

A cancer, however, is characterized by uncontrolled multiplication and disorganized growth of the affected cells. Concomitant with their capacity for unrestrained growth, cancer cells and the tissues they constitute lose their normal appearance, as viewed through a microscope, and assume aberrant functions. They are aggressive, invade surrounding tissues, spread to distant sites, and eventually kill the host. In 1994 cancers were responsible for almost one-fourth of all deaths in the United States.

Tumors, either malignant or benign, are based on the structural and functional properties of their component cells and their biological behavior. The cells and tissues of malignant tumors differ from the tissues from which they arise. They exhibit more rapid growth and altered structure and function. The properties of malignant tumor cells serve to enhance and support their proliferation and extension throughout the body tissues and organs. In contrast, the cells and tissues of benign tumors tend to grow more slowly and in general closely resemble their normal tissues of origin. When the structure and function of benign tumor cells are morphologically and functionally indistinguishable from those of normal cells, their growth as a tumor mass is the sole feature indicative of their neoplastic nature.

There is a general agreement within the art that the growth and duplication cycle of cells undergoing a continuous proliferation can be described as starting from a new small cell which passes through a first growth phase (G1). The G1 phase is followed by the phase of DNA synthesis, a copy of each chromosome being obtained (S phase). Then a second growth phase follows, whereby copies of all proteins, membranes, organelles, etc. are synthesized, which are needed in order to obtain two cells from the mother cell (G2 phase). During this G2 phase the dimensions of the cell increases. The mitotic phase (M phase) completes the actual cell division, two new cells being the result, and the cycle starts again.

A chemotherapy can cure certain forms of cancer, and chemotherapeutic agents have been used, which interfere with the cell cycle during mitosis, i.e. after the G2 phase. Such exogenous substances interfere with the assembling/disassembling kinetics of cytoskeletal proteins, e.g. tubulin fibers. Examples of such substances are for instance the vinca alkaloids (e.g. taxol).

Most cancer drugs, however, are limited in their usefulness. One problem is that only a certain proportion of cells is dividing at any one time, and most cancer drugs can destroy only that part of the cell population which undergoes division. Another problem is that cancer drugs, apart from damaging cancer cells, also damage normal cells and tissues. In order to overcome these difficulties, combinations of chemotherapeutic agents, which act on cells in different ways, have been used simultaneously or in sequence in various treatment programs.

The overall redox state of cells has been assumed to have relevance in the regulation of proliferation. Several observations report that when shifted to oxidation, cells are induced to proliferate, and when supplemented with reducing agents the cells tend to differentiate. Cells exposed to oxidative stress of various origin, i.e., UV radiation, ionizing radiation, and chemicals, often display a proliferative response. Free radicals and hydrogen peroxide residues cause the oxidative stress, and in most cases a pretreatment with antioxidants and free radical scavengers diminishes or completely abolishes the proliferative response.

N-acetyl-L-cysteine (NAC) is a thiol-containing compound which prevents or reduces the oxidation. Thus, it has several pharmacological and experimental uses. Although not finally proven, its properties are reasonably related to the sulfydryl residue (—SH). Due to its antioxidant properties, NAC has also been reported to act as an anti-inflammatory drug by counteracting the action of free radicals, which are byproducts of inflammation.

In WO 97/29759, NAC has been used in a pharmaceutical composition of doxorubicin for inhibiting the formation of cancer tumors. In this case, the antioxidant activity of NAC counteracts the cardiotoxicity of doxorubicin.

In Cell Proliferation (1998), 31(5/6), 217-229, thiol containing compounds exhibiting antioxidant properties have been evaluated for the use in cytoprotection and chemoprevention. N-Acetyl-L-cysteine (L-NAC) and its non-metabolically active stereoisomer N-acetyl-D-cysteine (D-NAC) were investigated together with the drugs captopril and dithioerythritol (DTT)in order to assess their effects on cell cycle progression. A decrease in topoisomerase-IIa activity was associated with a six-fold increase in the relative number of cells accumulating in the G2 phase.

Other pharmacological and clinical uses of NAC include the popular mucolytic action and the treatment of fulminant hepatic failure after treatment with paracetamol and related compounds. When used clinically, no significant side effects have been obtained with NAC, even after high doses, such as 10 g/day. This is opposed to other harmful thiol containing compounds, such as mercaptoethanol and dithiothreitol. Furthermore, the NAC clearance is relatively rapid.

In JP08040888 N-acetyl-L-cysteine (NAC) has been used as an anticancer agent for the treatment of myeloma type tumor because it has anticancer activity against myeloma type tumor cells in vitro, a tumor proliferation inhibitory activity being obtained.

In contrast to the myeloma type of tumors, the cell growth and cell renewal in most solid tissues of epithelial origin can be sketched as a duplication of a stem cell into one cell with an end-stage and one cell with a continued growth potential. The end-stage cell will then enter the differentiation pathway, in which it performs the diverse tissue functions, and it will have a finite life-span.

Cells entering the differentiation pathway exhibit a noticeable increase of cell-cell contacts, and the process is also generally indicated as contact inhibition. Several evidences indicate that signals for the cells to enter the differentiation end-point originate from the components of cell-cell contacts themselves. The cell-cell adhesions are also responsible for the diffusion of signals between cells.

Cancer cells can be regarded as differentiated cells that have lost this contact inhibition and that continuously proliferate. Thus, cancer cells have lost their ability to respond to differentiation signals. In this respect, instead of having the finite life-span of differentiated cells, cancer cells are eternal.

The purpose of the present invention is to produce a drug which acts as a therapeutic agent for those diseases which are related to a differentiation malfunctioning in epithelial tissues.

In order to achieve this purpose, the method according to the invention has been given the characterizing feature of claim 1.

It has surprisingly been discovered that N-acetyl-L-cysteine (NAC) and its disulfide dimer, N,N′-diacetyl-L-cystine (di-NAC), are reverting substances which without toxic effects revert the abnormal proliferation of neoplasia cells of epithelial origin back to a normal pathway, including block of proliferation, differentiation and a limited cell life-span. Other dimeric forms of N-acetyl-L-cysteine and similar structures, e.g. 4,4′-(isopropylidene-dithio)bis[2,6-di-tert-butylphenol], also exhibit these effects.

In this connection neoplasia cells means cells which form a neoplasm, i.e. the progressive multiplication of cells under conditions which would not elicit, or cause cessation of, multiplication of normal cells. Such a benign or malign hyperproliferation of cells encompasses metaplasia, i.e. the change in the type of cells in a tissue to a form which is not normal for that tissue; anaplasia, i.e. the loss of differentiation of cells and their orientation to each other; prosoplasia, i.e. the abnormal differentiation of a tissue; and retroplasia, i.e. the degeneration of a tissue or a cell into a more primitive type.

A reverting action of NAC, or di-NAC, involves the regular differentiation of cells in that the generally accepted differentiation in morphological heterogeneity occurs after the G1-G0 phases of the cell cycle and not at the G2 phase.

By using N-acetyl-L-cysteine, or a dimeric form thereof, according to the invention when a cell is differentiating—instead of dividing any further—the cell exhibits its final normal morphology. This reversion of the proliferating tumoral behavior in cells and tissues of epithelial origin is initiated before the new DNA synthesis, i.e. before the S phase. Then, from the G1 phase the cell enters the G0 phase (growth zero), from which the differentiation phase (D phase) starts. This phase results in the appropriate morphology, which is due to the synthesis and assembling of all components needed which all characterizes the finally differentiated and functionally active cell in the tissue.

The normal differentiation of neoplasia cells treated with NAC or di-NAC according to the invention can be demonstrated by the increase in cell-cell adhesion structures, the acquisition of several other structures as well as functions, which are typical for a well differentiated cell and differs for each cell type—as will be described below in the Examples—and by the arrest of proliferation. Other biochemical differentiation markers are early transcription factors.

The cell-cell adhesions obtained when for example a tumor is treated with high concentrations of NAC, or di-NAC, comprise several adhesive structures between adjacent cells and between cells and the basal extracellular matrix. These are in the microscope seen as tight and adherent junctions as well as desmosomes and gap junctions, each composed by a complex architecture of proteins, often assembled from repetitive sub-units. These contacts span the cell membrane and have extracellular, transmembrane and cytoplasmic sites. The cytoplasmic site of these multiprotein structures is, in turn, connected to the network of cytoskeletal proteins, while the extracellular site is connected either to a neighboring cell or to the basal extracellular matrix.

For example, two cell types, primary normal human epidermal keratinocytes (NHEK) and a human colon carcinoma line (CaCo2), both differentiate in 3 days when treated with NAC or di-NAC. The maximum inhibition of proliferation—which is demonstrated as no growth—is with these cells obtained at a local concentration of about 1 mM. Lower doses have also an anti-proliferative effect but not quite so extensive. A very low dose has a slightly proliferative effect due to the antioxidant properties of NAC and di-NAC. Thus, an effective dose of N-acetyl-L-cysteine, or a dimeric form thereof, which reverts mammalian neoplasia cells of epithelial origin to normal differentiation whereby the abnormal proliferation of such cells is reverted to a normal pathway, is according to the invention obtained at a concentration which is higher than that required for the maximum inhibition of proliferation, i.e. absolutely no growth. Accordingly, the inhibition of proliferation does not mean a reversion of neoplasia cells per se, but only if it is accompanied by a differentiation.

At these higher doses of NAC and di-NAC the breakdown of most cytoskeletal proteins takes place. NAC and di-NAC also prevents the natural restitution of the disrupted cytoskeleton. When the cytoskeleton is fragmented, stronger cell-cell contacts as well as an increased number of cell-cell junctions are obtained, i.e. an increased strength of the interactions between cell junctions and increased number of such junctions. This may be due to the cell-cell junctions being connected to the cytoskeleton network which keeps the junctions at a correct distance for the cell to proliferate. Once this net is broken by a sufficient concentration of NAC or di-NAC, the junctions will collapse and a tighter cell-cell contact will be obtained. This so-called contact inhibition results in a ceased proliferation, and surprisingly, the onset of differentiating, i.e. tumor reversion in the case of tumor cells. Thus, the reversion of malfunctioning mammalian cells of epithelial origin to a normal differentiation is induced through increased cell-cell contacts, a growth control of these cells being obtained.

Accordingly, the invention proposes the use of NAC, or di-NAC, for the clinical treatment of neoplasia cells of epithelial origin by inhibiting or stabilizing the corresponding disease, at high or low concentration, respectively. The type of neoplsia cells is not restrictive for the inventive use, and they can be anaplasia cells, metaplasia cells, prosoplasia cells, or retroplasia cells.

Preferably, the inventive use comprises an anticancer therapy, in which the tumor proliferation in tumors of epithelial origin is reverted into the differentiation pathway, including benign and malign hyperproliferation. Examples of benign tumor cells, which can be forced back by NAC, or di-NAC, into a normal differentiation, are epidermal tumor cells. and rheumatoidic cells. The malign tumors can be lung cancer, breast cancer, prostate cancer, or human colon carcinoma. A more comprehensive but non-limiting list of malign cells and tumors, which can be reverted back to a normal pathway according to the invention, includes the CNE2 human epithelial tumor cell line, the A431 cell line, 66-kDa Shc, esophageal cancer cell lines, the HepG2 human tumor cell line, human papillomavirus type 16-containing cancer cell lines, Merkel cell carcinoma cell lines, head and neck carcinoma cell lines, esophageal cancer cell lines, as well as glioblastoma cells, retinoblastoma, human retinoblastoma Y79 cells, hepatocellular carcinoma cells, SH-SY5Y cells, Ehrlich ascites tumor cells, liver epithelial tumor cells, spindle epithelial tumor with thymus-like differentiation (SETTLE), deciduoid epithelial mesothelioma, epithelial ovarian carcinoma, vulvar intraepithelial neoplasia, human mesenteric artery neoplasia, mesothelial neoplasia, intraepithelial neoplasia non-small-cell lung cancer, tubulocystic clear cell adenocarcinoma, colon adenocarcinoma cells, pigmented liver cell adenoma, non-colitic sporadic adenomas, Barrett's esophagus and associated adenocarcinoma, adenocarcinoma of the rectovaginal septum, prostate adenocarcinoma, pancreatic adenocarcinoma, pituitary acidophilic stem cell adenoma, salivary adenoid cystic carcinoma, adeno-endocrine cell carcinoma of the gallbladder, macrocystic serous cystadenoma of the pancreas, adenosquamous or columnar cell neoplasia, squamous cell carcinoma, cervical squamous cell carcinoma, neuroblastoma, squamous cell carcinoma arising from lesions of porokeratosis palmaris et plantaris disseminata, squamous-cell carcinoma of the cervix, solid-pseudopapillary tumor of the pancreas, renal cell carcinoma with or without rhabdoid features, hepatocellular carcinoma, large cell neuroendocrine carcinoma, small cell carcinomas, small cell lung cancer, small cell carcinoma of the breast, breast cancer, ductal carcinoma of the breast, signet ring cell lobular carcinoma, mucinous carcinoma, basal cell carcinoma cells, thyroid anaplastic carcinoma, carcinomas of the Waldeyer's ring area, hepatocellular-cholangiocarcinoma, glioma, C6 glioma cells, oligodendrogliomas and astrocytomas, gliosarcoma, myxopapillary ependymoma, pancreatic carcinoma cells, human prostate carcinoma cells, colorectal carcinoma, ovarian cancer cells, renal carcioma cells, non-melanoma skin cancers, parathyroid hormone-related protein and skin cancer, human laryngeal papilloma cells, acinic cell carcinoma of the oral cavity, Hurthle cell tumours of the thyroid, ovarian carcinomas, cell carcinoma of the bladder, colorectal cancer, enterochromaffin-like cells, human astrocytoma cells, human clear cell carcinomas of the kidney, metastatic renal-cell carcinoma, lingual carcinoma, head and neck adenopathy, large acinar cell carcinoma of the pancreas, hilar cholangiocarcinoma, papillary thyroid carcinoma, dent, human prostatic carcinoma cells, clear cell “sugar” tumors, myomelanocytic tumor of the falciform ligament/ligamentum teres (a member of the perivascular epithelioid clear cell family), and transitional cell carcinoma in the ureter.

Furthermore, keratosic skin, such as psoriasis and spongiotic dermatitis, can be effectively treated with NAC or di-NAC. Likewise, a correct epidermal tropism can be obtained with NAC or di-NAC, the endothelial cells of diabetic lesions formed in connection with a vascular damage being re-established.

It has been shown that di-NAC inhibits arteriosclerosis in WHHL rabbits. Likewise, renal epithelial tumors were found more often in kidneys with moderate and marked arteriolonephrosclerosis (40%) than in kidneys without or with minimal arteriolonephrosclerosis (8%) (Budin R E, McDonnell P J. Arch Pathol Lab Med 1984 February; 108(2):138-40). This association was found in patients both older and younger than 60 years. The development of renal epithelial tumors is associated with renal scarring; they are not independent age-related phenomena.

A reversion of 50% of arteriosclerotic cells to a normal pathway is supposed to be connected with cytokeratin expression. Expression of cytokeratins (CK) is considered a hallmark of the state of epithelial differentiation. CK also occur in certain vascular smooth muscle cells (VSMC), inferring an association with a less differentiated phenotype. CK posttranslational modification was shown to occur in epithelial cells in stress, mitosis, or apoptosis. Recently, CK phosphorylation patterns was investigated in human VSMC (Bar H, Bea F, Blessing E, Watson L, Wende P, Kreuzer J, Kubler W, Jahn L., Basic Res Cardiol 2001 February; 96(1):50-8).

Tissue samples of normal peripheral and coronary arteries, arteriosclerotic lesions and umbilical cord vessels were evaluated by immunofluorescence microscopy applying antibodies specific for cytokeratins 8 and 18, specific cytokeratin phosphorylation sites, Ki-67-antigen as a proliferation marker and nick end labeling (TUNEL) to detect apoptosis. All samples contained cytokeratin-positive VSMC but diverse phosphorylation patterns. The C-terminal serine 431 of cytokeratin 8 (CK8Ser-431) was phosphorylated in the vast majority of CK-expressing VSMC of coronary artery lesions. Only a subset of these cells demonstrated phosphorylation of CK18Ser-33 or, to an even lesser extent, CK8Ser-73.

DNA fragmentation occurred predominantly in samples containing cells with phosphorylated CK8Ser-431 domains. In contrast, occluded peripheral lesions exhibited little or no phosphorylation. Neonatal VSMC in umbilical cord vessels contain abundant phosphorylated CK domains, again predominantly CK8Ser-431, but also CK18Ser-33. Again, only single cells were found to be proliferating or to contain DNA fragmentation. Thus, abundant CK phosphorylation in VSMC of arteriosclerotic lesions suggests a specific functional response to cell stress and a possible relation to apoptosis.

By definition, all chemotherapeutic agents must be antiproliferative. Thus, the dose required in order to obtain a reverting effect of NAC, or di-NAC, according to the invention is higher than the dose required for inhibiting proliferation only. In contrast to all other chemotherapeutic substances reported, which are strongly toxic with enormous side effects, NAC can be used for treating neoplasia cells in local concentrations from 0.1 to 50 mM, depending on the type of cells treated, on the seriousness of the corresponding disease, and on the stage of the disease.

No toxic effects are obtained with the preferred concentrations between 0.25 and 40 mM NAC. For example, in order to rescue suicides after paracetamol poisoning NAC has been injected intravenously at a dose of 10 grams in 15 minutes, then half of the dose in the following 30 min, without any reported side effects at all.

Of course, the absolute maximum and minimum doses of NAC, or di-NAC, required for a complete block of proliferation depend on the cell type to be treated. For example, the minimum dose required in order to accelerate the differentiation of normal keratynocytes is 0.5 mM, and keratinocytes completely differentiate at 1 mM. The reversion of colon cancer cells is, however, induced at a concentration of NAC, or di-NAC, of 1 mM, and the colon carcinoma completely differentiate at 5 mM.

Furthermore, the differentiation effect obtained on colon carcinoma cells as well as normal keratinocytes is manifested directly, i.e. after only one supplementation of NAC or di-NAC.

Pharmaceutical and veterinary compositions may be prepared which contain effective amounts of NAC, or di-NAC, and a suitable carrier. Such carriers are well known to those skilled in the art. NAC or di-NAC may be administered directly or in the form of a composition to a human or animal subject.

The reverting substances according to the invention can intentionally be brought to a specific site of action by coupling the same to a targeting molecule, e.g. an antibody, which is directed towards an epitope in the vicinity of the site of action. Furthermore, NAC or di-NAC may be bound to antibodies directed towards a specific epithelial tissue in such a way that the reverting activity is targeted towards neoplasia cells of epithelial origin and fused with the same.

NAC or di-NAC can be supplemented locally as well as systemically and administrated orally, intravenously, intrarticularly, etc., depending on the type of the disease to be treated and on its stage. For example, for epidermal tumors a local application is sufficient. For rheumatoid arthritis, on the other hand, an intrarticular injection is needed. The appropriate route of administration also depends on the type of disease to be treated. For example, NAC has been effectively used for the local therapy of psoriasis as well as for the systemic and local therapy of psoriatic arthritis and of rheumatoid arthritis.

A treatment with NAC, or di-NAC, can be repeated several times with intervals, and the substance can be supplemented continuously as well. Since the tumor cells are not killed by this treatment, the time interval for the evident reversion of the disease depends on the specific life-span of the type of cells treated. With an epidermal tumor for example, having an epidermic cell life-span of about 30 days, an evident decrease of the tumor mass will appear after that time. When psoriasis is treated according to the invention, the symptoms disappear after treatments ranging from few days to 3-4 weeks, and the treatment may have to be repeated after a few months.

In the inventive use NAC, or di-NAC, not only initiates differentiating, i.e. tumor reversion in the case of tumor cells, by the growth control of malfunctioning cells which is induced through restored cell-cell contacts. At these concentrations NAC and di-NAC also exhibit the contact inhibition obtained at lower concentrations, which causes the cell to cease to proliferate. Furthermore, in the inventive use NAC and di-NAC also present the antioxidant effect.

### EXAMPLES

The invention will now be further described and illustrated by reference to the following examples. It should be noted, however, that these examples should not be construed as limiting the invention in any way.

### Example 1

### Effects of NAC and di-NAC on Human Colon Carcinoma Cells

An established cell line of human colon carcinoma cells was used (CaCo2). CaCo2 is a proliferating human colon carcinoma cell line displaying irregular morphology, forming multiple cell layers with scarce microvillous structures and large intercellular spaces.

After a long term culture of about 14 days the cells progressively acquire the typical morphology of differentiated cells, with

i) increased basolateral polarity,

ii) appearance of protruding villous structures at the upper surfaces of the cells (brush borders),

iii) localization of actin cytoskeletal fibers in the upper portions of the cells and of cytokeratin cytoskeletal fibers in the basal portions of the cells, which are connected to the newly established tight junctions with adjacent cells, and

iv) relocation of the nucleus closer to the basal membrane.

The CaCo2 cells were treated with various millimolar concentrations of NAC, ranging from 2 to 10 mM. NAC was added to the culture medium 24 hours after inoculation of the cells. After 3 days of culture in the presence of NAC the cells were analyzed for the following marker parameters:

Architecture of cytoskeleton: Disorganization of actin fibers was observed, with the appearance of stress fibers. Relocalization of actin fibers in the upper portion of the cell, sustaining the brush border microvilli, and relocalization of the cytokeratin fibers in the basal portion of cell. Cytokeratin fibers also appeared sparsely fragmented.

Surface morphology: A relevant number of microvillous structures was observed at the cell surface, having the morphology of end-stage differentiated cells.

Cell-cell adhesion: An increased number of all kinds of cells adhesion structures was observed. The intracellular space was also dramatically reduced.

Tubulin expression and polymerization: The tubulin expression appeared strongly reduced by the NAC treatment. The mitotic spindle was often absent in cells in the mitotic metaphase.

Proliferation: The cell proliferation decreased in a dose-dependent fashion, with a maximum effect at 2 mM NAC.

Toxicity: The cell viability was unaffected by NAC treatment at all doses added. Neither apoptosis nor necrosis was observed.

Likewise, a treatment of CaCo2 cells with di-NAC induces morphological and biochemical changes in the cells. For example increased cell thickness, apical-basolateral polarity, increased E-cadherin expression, formation of brush border and tight junctions as well as a decreased proliferation is obtained. In some cases the treatment with di-NAC was more effective than the treatment with NAC.

In summary, when treated with NAC or di-NAC, the CaCo2 cells are induced to revert to a differentiation pathway without any detectable toxic side effects. As a consequence of their differentiation the proliferation of the CaCo2 cells is also decreased.

### Example 2

### Effects of NAC or di-NAC on Human Epidermal Keratinocytes

Primary normal human epidermal keratinocytes (NHEK) obtained from Clonetics Inc. (San Diego, Calif., U.S.A.) were cultivated in the medium provided by the supplier. These cells have a limited life-span of about 15 days in culture and undergo a normal differentiation process of epidermal keratinocytes with a final enucleation and exfoliation. After inoculation the cells acquire a polygonal morphology cell-cell adhesions. The cytoskeleton morphology is quite organized, mainly at the cell borders.

NHEK cells were treated with various millimolar concentrations of NAC or di-NAC, ranging from 1 to 10 mM. The substances were added to the culture medium 24 hours after inoculation. After 3 days of culture in the presence of 2 mM of NAC or di-NAC the cells were analysed for the following marker parameters:

Architecture of cytoskeleton: Disorganization of actin fibers was observed, that appeared dispersed all over the cell cytoplasm and several stress fibers appeared. Cytokeratin fibers were dramatically fragmented. The cytoskeleton fragmentation was also monitored by means of electrophoresis. Several bands were obtained after the treatment, while only one broad band was visible with untreated cells.

Surface morphology: The cell surface appeared smooth with the loss of the fine microvillous structure which is representative of proliferating keratinocytes. A significant number of enucleated cells was observed concomitantly with the appearance of the initial stages of the cornified envelope.

Cell-cell junctions: An increased number of junctions was observed, and the intracellular space was dramatically reduced. The concentration of E-cadherins was also increased.

Tubulin expression and polymerization: Tubulin expression appeared to be reduced by the treatment. A small reduction in percent of the formation of the mitotic spindle was observed in cells in the mitotic metaphase.

Differentiation: The disappearance of markers for the proliferative state of keratinocytes was determined by Western blotting.

Proliferation: The cell proliferation decreased in a dose-dependent fashion with the complete suppression of cell proliferation at 2 mM of NAC or di-NAC.

Toxicity: The cell viability was unaffected by NAC treatment at all doses, i.e., up to 10 mM. Neither apoptosis nor necrosis was observed.

It should be pointed out that the isolated keratynocytes were treated with NAC in the absolute absence of lymphocytes.

In summary, the treatment of NHEK cells with of NAC or di-NAC induced reversion and a 3-4 fold faster differentiation with no toxic side effects. The induced differentiation also resulted in that the NHEK proliferation was abolished.

### Example 3

### Effects of NAC or di-NAC on Psoriasis

Psoriasis is a chronic skin disorder which is distinguished by a benign hyperproliferation of cells, a production of cytokines, an accumulation of inflammatory cells, an abnormal keratinization, and an increased vascularization.

Five informed volunteers displaying localized psoriatic plaques were locally treated with a base-cream containing 10-20 weight % of NAC or di-NAC. The cream had been applied every night for variable periods—from weeks to a few months—with or without occlusive bandage. The skin of the patient was normally washed between each daily treatment. No other drugs were contemporarily used.

A rapid improvement was observed already after 1-2 treatments with decreased scales as well as skin smoothing. A prolonged treatment resulted in a complete remission of all symptoms. The length of the treatment required for a complete remission varied depending on the individual response and on the seriousness of the disease. The most commonly reported side effect concerned a local hitching which disappeared when the occlusive bandage was removed. In one case, swelling was also reported.

After the treatment was discontinued, only one patient exhibied new small psoriatic plaques after 4 months, which disappeared after a few days further treatment.

Thus, the abnormal proliferation as well as the lack of differentiation of psoriatic keratynocytes were both abolished.

### Example 4

### Effects of NAC on Large Intestinal Tumors

The effects of daily NAC treatment or intermittent NAC treatments on intestinal tumors, which had been induced by treating male Sprague-Dawley rats with 1,2-dimethylhydrazine (DMH), were investigated.

The large intestinal tumor burden was significantly lower for rats intermittently treated with NAC than for a corresponding control group (11 and 1 tumors/treatment group, respectively, p=0.001). The large intestinal tumor mass index was likewise significantly lower for the group treated with NAC than for control group (1.93 and 0.04, respectively, p<0.001). For rats treated with NAC daily, no adenocarcinomas were present in the colons.

It is concluded that NAC reduces the DMH-induced large intestinal tumors in male Sprague-Dawley rats and has a protective effect as seen by the significant inhibition of tumors within the rat colon.

### Example 5

### Effects of NAC on Colon Adenocarcinoma

In this experimental model BD-IX rats were divided into four groups: Groups G1 and G2, designated “cancer groups”, were used to study the effects of NAC on the progression of colon cancer, and Groups G3 and G4, designated “toxicity groups”, were used to study the effects of the treatment on metabolic processes and the parenchyma. DHD/K12-PROb cells were injected subcutaneously into the chest of Group G1 and G2 animals. From 1 to 13 weeks after inoculation the animals in Groups G2 and G4 received a weekly injection of NAC. The animals in Groups G1 and G3 received no treatment.

In addition, lines of animal and human colon adenocarcinoma cells (DHD/K12-PROb and HT-29) were used to perform assays in vitro in order to examine the cytotoxicity of NAC.

It was found that a long-term treatment of colon adenocarcinoma with NAC in this experimental study in the rat resulted in an extended life span. Furthermore, the tumor growth was significantly slower in female as well as male rats, which had been treated with NAC.

### Example 6

### Effects of NAC on Colon Cancer, Mechanisms of Chemoprevention

Since the efficacy of NAC against colon cancer is significant, the mechanisms of inhibiting colon carcinogenesis were explored by investigating the activities of protein kinase C (PKC), tyrosine protein kinase (TPK), and diacylglycerol kinase (DGK) as well as the 8-isoprostane levels in colonic mucosa and tumor tissues in an azoxy-methane (AOM)-induced rat colon cancer model.

At seven weeks of age corresponding groups of rats were injected s.c. with azoxymethane (AOM; 15 mg/kg body weight, once weekly for 2 weeks), and different experiments were continued until 38 weeks after the second AOM treatment. Then, the rats were then sacrificed and colonic mucosal as well as tumor samples were evaluated for PKC, TPK, DGK, and 8-isoprostane levels.

The administration of NAC significantly inhibited Ca²⁺ dependent and independent PKC (P<0.01) activities in colonic mucosa and tumors. The administration of NAC either significantly suppressed both colonic mucosal and tumor TPK activities (P<0.01). In contrast, rats which had received NAC, exhibied a significant increased DGK activity (P<0.01). In addition, rats treated with NAC had lower levels of 8-isoprostane in the colonic tumors than the control.

These findings suggest that the mechanisms of NAC in the chemoprevention of colon cancer involves the modulation of protein kinase C, tyrosine protein kinase and diacylglycerol kinase activities by inducing down-regulation of the PKC and TPK activities and up-regulation of the DGK activity. Thus, these events may in part be responsible for the chemopreventive activity against colon carcinogenesis.

Furthermore, these results imply that NAC will augment the regulation of PKC, TPK and DGK activities in the colon.

### Example 7

### Effects of NAC or di-NAC on Oral Keratinocytic Tumors and Their Metastatic Dissemination

The effects of NAC or di-NAC on the metastatic capacity of clonal populations of 4NQO-induced rat malignant oral keratinocytes were examined after orthotopic transplantation to athymic mice. Polygonal cells and spindle cells formed well-differentiated squamous cell carcinomas (keratin positive and vimentin negative) and undifferentiated spindle cell tumors (keratin negative and vimentin positive), respectively, in all animals at the site of inoculation (floor of mouth).

The transplantation of 5×10⁶ cells of either cell type at high cell density resulted in that approximately 50% of the animals formed pulmonary metastases.

Undifferentiated spindle cell lines expressed transforming growth factor β1 (TGF-β1) and, following transplantation orthotopically and treatment according to the invention, fewer animals formed pulmonary metastases despite the formation of primary tumors in almost all grafted animals.

The results suggest that the TGF-β1 induced by NAC, or di-NAC, may act as a tumor suppressor in this cell type. The clones of polygonal cells were markedly inhibited by exogenous NAC, and the spindle cells were inhibited as well. The results also suggest that differentiated rat malignant oral keratinocytes are less aggressive when treated with NAC or di-NAC, and they have a decreased potential to metastasise than their untreated counterparts. Similar findings were seen in the undifferentiated spindle cell counterparts. The contribution of TGF-β and its receptor profile may partly account for the finding that NAC or a dimeric form thereof reduces malignant oral keratinocytic tumors and their metastatic dissemination.

### Example 8

### Tissue Targeting

Influenza virosomes (reconstituted influenza virus envelopes) can be targeted towards ovarian carcinoma cells (OVCAR-3) with preservation of fusion activity. This is achieved by incorporating poly(ethylene glycol) (PEG)-derivatized lipids into the virosome membrane (Mastrobattista E, Schoen P, Wilschut J, Crommelin D J, Storm G. FEBS Lett Nov. 30, 2001; 509(1):71-76).

Fab′-fragments of the monoclonal antibody (mAb) 323/A3 (anti-epithelial glycoprotein-2) were coupled to the distal ends of PEG lipids. This PEG layer serves as shield to prevent interaction of viral hemagglutinin with ubiquitous sialic acid residues and as spatial anchor for antibody attachment. As a result, a specific binding of virosomes OVCAR-3 cells is obtained. The antibody-redirected virosomes fuse with membranes of OVCAR-3 cells in a pH-dependent fashion.

By using such a procedure (Mastrobattista E, Schoen P, Wilschut J, Crommelin D J, Storm G., FEBS Lett Nov., 30, 2001; 509(1):71-76) NAC and di-NAC can be coupled to Fab′ fragments of mAb 323/A3 and attached to virosomes.

These experiments demonstrate that NAC and DiNAC can be used in connection with tissue targeting.

### Example 9

### Inhibition of PPAR Synthesis by NAC Treatment

Peroxisome proliferator-activated receptors (PPARS) are ligand-activated transcription factors belonging to the nuclear receptor family (Chinetti G., Fruchart J.-C., Staels B., Inflammation Research Abstract Volume 49 Issue 10 (2000), pp 497-505). These receptors function as regulators of lipid and lipoprotein metabolism and glucose homeostasis and influence cellular proliferation, differentiation and apoptosis. PPAR-α is highly expressed in tissues such as liver, muscle, kidney and heart, where it stimulates the β-oxidative degradation of fatty acids. PPAR-δ is predominantly expressed in intestine and adipose tissue. PPAR-γ triggers adipocyte differentiation and promotes lipid storage. The hypolipidemic fibrates and the antidiabetic glitazones are synthetic ligands for PPAR-α and PPAR-γ, respectively. Furthermore, fatty acids and eicosanoids are natural PPAR ligands: PPAR-α is activated by leukotriene B4, whereas prostaglandin J2 is a PPAR-γ ligand.

Furthermore, PPAR-α deficient mice display a prolonged response to inflammatory stimuli. PPAR activators have also been shown to inhibit the activation of inflammatory response genes (such as IL-2, IL-6, IL-8, TNF-α and metalloproteases) by negatively interfering with the NF-κ, STAT and AP-1 signalling pathways. PPAR activators exert these anti-inflammatory activities in different immunological and vascular wall cell types, such as monocyte/macrophages, endothelial, epithelial and smooth muscle cells, in which PPARs are expressed.

Effective concentrations of NAC (c.f. above) thus exhibit not only a metabolic but also an inflammation control by inhibiting PPARs.

Potential therapeutic applications are inflammation-related diseases, such as atherosclerosis and inflammatory bowel disease.

1. Use of an effective dose of N-acetyl-L-cysteine, or a dimeric form thereof, for the manufacturing of a drug for reverting mammalian neoplasia cells of epithelial origin back to a normal differentiation, whereby the abnormal proliferation of the neoplasia cells is reverted to a normal pathway.

2. Use as in claim 1, characterized in that the neoplasia cells are anaplasia cells.

3. Use as in claim 2, characterized in that the anaplasia cells are benign tumor cells.

4. Use as in claim 3, characterized in that the benign tumor cells are epidermal tumor cells.

5. Use as in claim 4, characterized in that the epidermal tumor cells are psoriatic cells.

6. Use as in claim 5, characterized in that the benign tumor cells are rheumatoidic cells.

7. Use as in claim 2, characterized in that the anaplasia cells are malign tumor cells.

8. Use as in claim 7, characterized in that the malign tumor cells are lung cancer, breast cancer, prostate cancer, or human colon carcinoma.

9. Use as in claim 1, characterized in that the neoplasia cells are metaplasia cells.

10. Use as in claim 9, characterized in that the metaplasia cells are keratosic cells.

11. Use as in claim 10, characterized in that the keratosic cells are psoriatic cells.

12. Use as in claim 9, characterized in that the metaplasia cells are cells exhibiting diabetic lesions.

13. Use as in claim 1, characterized in that the neoplasia cells are prosoplasia cells.

14. Use as in claim 1, characterized in that the neoplasia cells are retroplasia cells.

15. Use as in any of claims 1-14, characterized in that the effective dose of N-acetyl-L-cysteine, or a dimeric form thereof, is locally from 0.1 to 50 mM.

16. Use as in any of claims 15, characterized in that the effective dose of N-acetyl-L-cysteine, or a dimeric form thereof, is locally from 0.25 to 40 mM.

17. A composition for reverting mammalian neoplasia cells of epithelial origin back to a normal differentiation whereby the abnormal proliferation of the neoplasia cells is reverted to a normal pathway, characterized by a therapeutically effective amount of N-acetyl-L-cysteine, or a dimeric form thereof, and a pharmaceutically acceptable carrier.

18. A composition as in claim 17, characterized in that a tissue targeting molecule is attached to N-acetyl-L-cysteine or its dimeric form.
